SEQUENCE LISTING (4 sequences)

SEQ ID NO: 1 - DNA, 36 bp, Artificial
  CIORfwd2 primer for inverting amplifier genetic circuit, selects lambda prm cI
gcgcgcgcgaattccttgccgatcagccaaacgtct

SEQ ID NO: 2 - DNA, 35 bp, Artificial
  CIOR-NcoI primer for inverting amplifier genetic circuit, selects Lambda prm cI
gcgcgcgccatggcaaccattatcaccgccagagg

SEQ ID NO: 3 - DNA, 47 bp, Artificial
  Primer for inverting amplifier genetic circuit, selects EGFP
gcgcgcgaattcaggaggtaacagctcatggtgagcaagggcgagga

SEQ ID NO: 4 - DNA, 36 bp, Artificial
  Primer for inverting amplifier genetic circuit, selects EGFP
cgcgcggattccttacttgtacagctcgtccatgcc

(19) United States
(12) Patent Application Publication
(10) Pub. No.: US 2005/0112615 A1
(43) Pub. Date: May 26, 2005

(54) Genetic circuit inverting amplifier

(76) Inventors: Stephen William Davies, Toronto (CA); Gianna DeRubertis, Montreal (CA)

Correspondence Address: STEPHEN WILLIAM DAVIES, 4 Taddle Creek Rd., RS-407, TORONTO, ON, M5S 2S3, CA

(21) Appl. No.: 10/861,415

(22) Filed: Jun. 7, 2004

Related U.S. Application Data
(60) Provisional application No. 60/476,120, filed on Jun. 5, 2003.

(51) Int. Cl. C12Q 1/68; H03F 21/00

(52) U.S. Cl. 435/6; 330/207 R

(57) ABSTRACT

We describe methods and compositions for setting the level of gene expression in a cell. The instant invention is of an amplifier, an analog circuit. It is directly analogous to the inverting amplifier commonly used in electronics. This circuit produces an output wherein an increase in the input signal leads to a proportional decrease in the output. Similarly, a decrease in the input leads to an increase in the output signal. Here the inputs and outputs may be concentrations of intracellular molecular species. The circuit is implemented as nucleic acid constructs. By analogy with the similar circuit found in analog electronics, such a circuit is expected to have improved signal to noise ratio, improved stability, and increased accuracy. It should aid in the development of intracellular instrumentation and hence the diagnosis and treatment of disease.

### CROSS-REFERENCE TO RELATED APPLICATIONS

This application claims priority to, and the benefit of U.S. Ser. No. 60/476,120 filed on Jun. 5, 2003, the disclosures of which are incorporated by reference herein.

### STATEMENT REGARDING FEDERALLY SPONSORED RESEARCH

United States federal sponsorship was not involved in this work.

### REFERENCE TO A SEQUENCE LISTING

An appendix provides listing of DNA sequences used in the construction of the circuit.

### BACKGROUND OF THE INVENTION

### References

1. Becskei, A., Serrano, L., “Engineering stability in gene networks by autoregulation”, Nature 405, 1 Jun., 2000, pp.590-593.
2. McAdams, H. H., Arkin, A., “Gene regulation: towards a circuit engineering discipline”, Current Biology, Vol.10, No.8, 2000, pp.R318-R320.
3. Kennell, D., Riezman, H., “Transcription and translation initiation frequencies of the Escherichia coli lac operon”, J. Mol. Biol., Vol.114, 1977, pp.1-21.
4. McClure, W. R., “Rate-limiting steps in RNA chain initiation”, Proc. Natl. Acad. Sci. USA, Vol.77, No.10, October, 1980, pp.5634-5638.
5. Yarchuk, O., Jacques, N., Guillerez, J., Dreyfus, M., “Interdependence of translation, transcription and mRNA degradation in the lacZ gene”, J. Mol. Biol., Vol.226, 1992, pp.581-596.
6. Lombardi, L., Ciana, P., Cappellini, C., Trecca, D., Guerrini, L., Migliazza, A., Maiolo, A. T., Neri, A., “Structural and functional characterization of the promoter regions of the NFKB2 gene”, Nucleic Acids Research, Vol.23, No.12, 1995, pp2328-2336.
7. Keller, A. D., “Model genetic circuits encoding autoregulatory transcription factors”, J. theor. Biol., Vol. 172, 1995, pp. 169-185.
8. McAdams, H. H., Arkin, A., “Stochastic mechanisms in gene expression”, Proc. Natl. Acad. Sci. USA, Vol.94, February, 1997, pp.814-819.
9. Little, J. W., Shepley, D. P., Wert, D. W., “Robustness of a gene regulatory circuit”, EMBO J., Vol.18, No.15, 1999, pp.4299-4307.
10. Gardner, T. S., Cantor, C. R., Collins, J. J., “Construction of a genetic toggle switch in Escherichia coli”, Nature, Vol. 403, 20 Jan., 2000, pp.339-342.
11. Elowitz, M. B., “A synthetic oscillatory network of transcriptional regulators”, Nature, Vol.403, 20 Jan., 2000, pp.335-338.
12. Maloy, S. R., Cronan, J. E., Freifelder, D., “Microbial genetics”, Jones and Bartlett, Sudbury, Mass., USA, 1994, pp.131-133.
13. Sambrook, J., Russell, D. W., “Molecular Cloning: A Laboratory Manual, 3^{rd} ed. Cold Spring Harbour Laboratory Press, Cold Spring Harbour, N.Y., 2001.
14. Lutz, R., and Bujard, H., “Independent and tight regulation of transcriptional units in Escherichia coli via LacR/O, the TetR/O and AraC/I1-I2 regulatory elements”, Nucleic Acids Research Vol. 25, 1997, pp. 1203-1210.
15. “Tools and technology for life sciences”, Stratagene Corp., LaJolla, Calif., USA, 2001, pp.40-41.
16. De Rubertis, G., Davies, S. W., “Genetic circuit design from an electronics perspective”, IEEE Conf. Molecular, Cellular and Tissue Engineering, Genoa, Italy, 6-9 Jun. 2002, pp.123-124.
17. De Rubertis, G., Davies, S. W., “Design and simulation of a genetic circuit amplifier”, 10th Intl. Conf. on Intelligent Systems for Molecular Biology, Edmonton, Canada, Aug. 3-7, 2002, pg.113.
18. DeRubertis, G., M.A.Sc. thesis, University of Toronto, September, 2002.
19. Seale, D., Davies, S. W., “Stochastic model of DNA microarrays”, IEEE Conf. Molecular, Cellular and Tissue Engineering, Genoa, Italy, 6-9 Jun. 2002, pp.113-114.
20. Seale, D., Davies, S. W. “Performance analysis of an optimal estimator of gene expression ratio”, 10th Intl. Conf. on Intelligent Systems for Molecular Biology, Edmonton, Canada, Aug. 3-7, 2002, pg.55.
21. Seale, D., Davies, S. W., “Optimal estimation of gene expression”, IEEE 2nd Joint EMBS-BMES Conf., Houston, USA, 23-26 Oct., 2002.
22. MacIsaac, K., Davies, S. W., “Maximum likelihood estimation of rate parameters in single-molecule observable biological systems”, 27th Canadian Medical and Biological Engineering Conference, Ottawa, Ontario, Nov. 21-23, 2002.
23. Sedra, A. S., Smith, K. C., “Microelectronic circuits”, 4^{th} ed., Oxford University Press, New York, N.Y., USA, 1982, pp. 64-68.
24. Ptashne, M., “A genetic switch”, 2ed., Blackwell Science Inc., Malden, Mass., USA, 1992.
25. Kay, M. A., Glorioso, J. C., Naldini, L., “Viral vectors for gene therapy: the art of turning infectious agents into vehicles of therapeutics”, Nature Medicine, Vol. 7, No. 1, January, 2001, pp.33-40.
26. Clarkson, T., “Regulated gene expression systems”, Gene Ther., Vol. 7, 2000, pp.120-125.
27. Winter, P. C., Hickey, G. I., Fletcher, H. L., “Genetics”, 2^{nd} Ed., BIOS Scientific Publishers, Oxford, UK, 2002, pp.57-58.
28. Wagner, R., “Transcription Regulation in Prokaryotes”, Oxford University Press, Oxford, UK, 2000.
29. Yokbayashi, Y., Weiss, R., Arnold, F. H., “Directed evolution of a genetic circuit”, PNAS early edition, www.pnas.org/cgi/doi/10.1073/pnas.252535999, 2002, pp.1-5.
30. Leung, D. E., Chen, E., Goeddel, D. V., “A method for random mutagenesis of a defined DNA segment using a modified polymerase chain reaction”, Technique, Vol. 1, pp.11-15.
31. Shimizu-Sato, S., Huq, E., Tepperman, J. M., Quail, P. H.,“A light-switchable gene promoter system”, Nature Biotech., Vol. 20, No. 10, 2002, pp.1041-1044.
32. Weiss, R., “Cellular Computation and Communications using Engineered Genetic Regulatory Networks”, Ph.D. Thesis, M.I.T., Boston, August 2001
33. Yao, F., Eriksson, E., “A novel tetracycline-inducible viral replication switch”, Human gene therapy, Vol. 10, No. 3,Feb. 10, 1999, pp.419-27.
34. Baron, U., Gossen, M., Bujard, H., “Transcriptional activators with graded transactivation potential”, U.S. Pat. No. 6,271,341, Aug. 7, 2001.
35. Arkin, A. P., “Signal processing by biochemical reaction networks”, in Self Organized Biological Dynamics & Nonlinear Control, Walleczek, J., Editor,Cambridge University Press, Cambridge, UK, 2000,pp.112-144.
36. Kim, D. H., McCormick, F., “Replicating viruses as selective cancer therapeutics”, Mol. Med. Today, Vol. 2, 1996, pp.519-527.
37. Ramachandra, M., et al, “Re-engineering adenovirus regulatory pathways to enhance oncolytic specificity and efficacy”, Nature Biotech., Vol. 19, No. 11, 2001, pp.1035-1041.

A genetic circuit is an intriguing way to view the signaling and feedback pathways in a cell. Here, the signaling molecules are seen as the wires in the circuit, connecting each output to another input. The phrase “genetic circuit” actually dates from the early 1960's but it has acquired new vitality as we enter the infancy of genetic circuit engineering [2]. In the intervening period, existing circuits in living creatures were deduced. Researchers measured and characterized the behaviors (e.g. binding constants) of components of genetic circuits [3-6]. Theoretical models of regulatory genetic circuits were developed [7-9]; in particular, these models addressed the molecule-by-molecule stochastic nature of gene control circuits. In the same period, the tools of genetic engineering, such as cloning [13], were developed. Recently, synthetic genetic circuits have been created that demonstrate a number of basic engineering functions. A toggle switch, a regulator and an oscillator have been reported.

The “toggle switch” is a digital genetic circuit with outputs being either on or off[10]. This device enters the “on” state when a certain input signal (isopropyl-β-D-thiogalactopyranoside(IPTG)) is applied and remains in that state when the input is removed. Applying a different kind of input signal (anhydrotetracycline(aTc) or heat, depending on the promoter used) sends it to the “off” state where it remains when said input is removed. The IPTG inducer interacted with lacI repressor and promoter Ptrc-2, all basically from the lactose operon, the operon explained first in microbial genetics texts. The aTc inducer interacted with repressor tetR and promoter P_{L}tetO-1. A gene for green fluorescent protein (gfp) was used to produce the output signal. The plasmid was inserted into an E. coli strain.

The regulator, an analog genetic circuit, is analogous to the voltage or current regulator found in electronic circuits. The investigation centered on the variation of the preset output level in the face of external disturbances [1]. The plasmid implementation centered on the commonplace repressor (tetR) for tetracycline resistance and its corresponding operator sequence. A gene for green fluorescent protein (gfp) was used to produce the output signal. The plasmid was inserted into an E.coli strain.

Using similar components, an oscillator has been created [11]. This “Repressilator” features three promoters, for CI, TetR and LacI, connected in a circle. An active promoter 1 turns off the promoter 2 at its output which in turn allows the promoter 3 following that one to turn on and suppress promoter 1. Thus a wave of activity cycles through the promoters in reverse order.

Weiss employed random generation and selection to produce circuits with different combinatorial logic functions [32].

Commercial sources have advanced to where they are now supplying two plasmid systems for studying interactions between two proteins. Plasmids providing intracellular protein output proportional to a membrane diffusable agent (e.g. IPTG) are also available [15].

Both analog and digital functions are under investigation in the applicants' laboratory [16-18]. In addition to the instant invention, a genetic circuit memory element has been developed. In standard instrumentation, analog and digital electronics are integrated together to provide an instrumentation system. Our work has applied optimal signal processing techniques to molecular biological instrumentation problems[19-22]. In general, our work in genetic circuits seeks to integrate analog and digital functional blocks to provide a living instrumentation system.

### SUMMARY OF THE INVENTION

This invention concerns implementation of what is known as a genetic circuit, which is a set of genes engineered to perform one or more signal processing functions, much like electronic circuits do. The instant invention is of an amplifier, an analog circuit. It is directly analogous to the inverting amplifier commonly used in electronics. This circuit produces an output wherein an increase in the input signal leads to a proportional decrease in the output. Similarly, a decrease in the input leads to an increase in the output signal. Here the inputs and outputs are concentrations of intracellular molecular species.

Synthetic genetic circuits have been created that demonstrate a number of basic engineering functions. A toggle switch, a regulator and an oscillator have been reported. Combinatorial logic circuits have been described, including a logical inverter. The logic inverter is a digital circuit wherein a high input leads to a low output and vice versa. The key innovation in the instant invention is to use negative feedback to counter the influence of the input signal. This allows the change in output to be more proportionate to the change in input.

As proven in analog electronic circuits, amplifiers employing negative feedback offer several advantages over systems without feedback. These include improved signal to noise ratio, improved stability, and increased accuracy. Intracellular genetic circuits based on this technology may aid investigations into cell differentiation and cancer.

### BRIEF DESCRIPTION OF THE DRAWINGS

FIG. 1 is a schematic illustration of the typical electronic circuit implementation of an inverting amplifier.

FIG. 2 is a schematic illustration of the P_{RM} promoter region of bacteriophage λ

FIG. 3 is a schematic illustration of the biological design of the genetic circuit amplifier.

### DETAILED DESCRIPTION OF THE INVENTION

The instant invention is of an amplifier, an analog circuit. It is directly analogous to the inverting amplifier commonly used in electronics. This circuit produces an output wherein an increase in the input signal leads to a proportional decrease in the output. Similarly, a decrease in the input leads to an increase in the output signal. Here the inputs and outputs are concentrations of intracellular molecular species. The key innovation is to use negative feedback to counter the influence of the input signal. This allows the change in output to be more proportionate to the change in input.

### 1. Inverting Amplifier Function and Structure

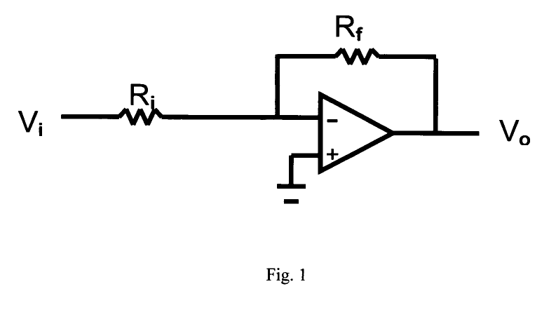

FIG. 1 shows a typical electronic circuit implementation of an inverting amplifier [23]. There are three components in this particular implementation. Two of these components are resistors while the third is an operational amplifier. An operational amplifier is a gain element wherein the output is a very large multiple, A, ideally an infinite multiple, of the difference between its positive and negative inputs. Resistor Ri couples the circuit input, Vi, to the negative input of the operational amplifier. Resistor Rf couples the circuit output, Vo, to the negative input of the operational amplifier.

The positive input is tied to ground. The operational amplifier responds to the difference between its positive V₊ and negative V₋ inputs. However for this grounded configuration, V₊=0. Thus the difference is V₊−V₋=0−V₋=−V₋. Thus, the operational amplifier is in a sense reduced to having a single negative input. It output is then −AV₋.

In the circuit of FIG. 1, the feedback of the output through Rf acts to cancel the contribution of the input. In the case where the gain element has infinite gain and Rf=Ri, equilibrium is achieved when Vo=−Vi. Overall circuit gain is defined as Vo/Vi so the gain in that case is −1. For arbitrary values of the resistors, the overall gain can be shown to be −Rf/Ri. If the gain element has less than infinite gain [23], the overall circuit gain becomes −(Rf/Ri)(1/(1+(Rf/Ri)/A)). This is essentially −Rf/Ri as long as A is much greater than Rf/Ri. Thus this feedback allows the circuit designer to set the circuit gain to be −Rf/Ri independent of what the gain of the underlying gain element is so long as that gain element has high gain.

Many variants of this design are possible. Inverting amplifiers are often created using less than ideal gain elements. For example, the operational amplifier with positive input tied to ground may be replaced by a bipolar transistor and additional resistor(s) added to bias the transistor into a particular part of its operating characteristic. This transistor is likely to have lower gain than an operational amplifier and its input output relation is non-linear. However, the action of the negative feedback in the resulting inverting amplifier is to reduce the impact of the non-linearity, leading to a more faithful, albeit inverted, reproduction of the input signal with gain largely determined by the choice of resistors. Thus, we are motivated to implement a similar design in a genetic circuit wherein the gain element (e.g. a promoter) may be of low gain and highly non-linear; the expectation is that the circuit would be more linear and more predictable than in the case without feedback.

### 2. Genetic Circuit Gain Element

The essential requirements the gain element is that its output should be a decreasing function of its input. A desirable feature of the gain element is that the magnitude of the slope of this function be large. This corresponds to high gain.

A repressible promoter satisfies the essential requirement. Here the repressor molecule serves as the input to the gain element. When a repressor molecule binds to the operator site upstream of the promoter, it suppresses expression of the associated gene. Increasing the repressor concentration would then reduce the frequency at which new transcripts are started at the promoter. As appropriate to the design, the output may be defined to be the mRNA transcript, the protein obtained by translating the transcript, or some other molecule that is produced as a result of producing that protein. Thus the output would be a decreasing function of the input.

Alternatively, a more complicated design may be obtained by further exploiting the analogy with electronics. In electronics, an operational amplifier may have complicated internal circuitry encompassing many transistors. However, its use as a gian element depends only on the relation of its input to its output, and in particular that the output is a decreasing function of its negative input. Any molecular pathway where the output of that pathway is a decreasing function of the input to that pathway may be used. This pathway might include metabolic steps and/or multiple coupled promoters.

Of course, the presumption is that a means of coupling the output molecules to the input is available. For the simple circuit based on a single repressible promoter, the gene attached to the promoter could code for the repressor. For the more complicated design, a promoter at the end of the pathway could be attached to a gene that codes for a molecule that will ultimately lead to a change in concentration of the input molecule. This might be direct in that the gene codes for the protein that is the input molecule, or less direct in the that the protein is a substrate for the production of the input molecule, or still less direct in that the protein modulates the production of the input molecule.

It is not necessary that the output be a monotonically decreasing function of the input throughout the entire range of inputs. Rather it is a requirement that the gain element have at least one region where the output is a monotonically decreasing function of the input and that the circuit provides a means of operating the gain element over just that range. In electronics, this means is usually through providing a bias current that puts the gain element in the desired operating region. The input is then seen as being relative to that bias current.

### 3. Bias

Bias voltages and/or currents are widely used in electronic circuitry. As mentioned in the previous section, bias may be used to place the gain element within the desired operating region. Bias may also be used to provide a reference about which the signal is defined. This is particularly true when the power supply of the circuit only provides a single voltage with respect to ground. For example, a circuit powered +10 volt power supply cannot provide a negative output. Instead the designer might bias its output to provide +5 volts for zero input voltage. Then a negative output could be defined relative to this bias level. Thus when the circuit outputs 4 volts it should be interpreted as a negative output, −1 volt relative to the bias level. Similarly, the input circuitry can be biased to allow representation of negative inputs.

For the genetic circuit inverting amplifier, the inputs and outputs are likely concentrations of molecular species and are therefore always greater than or equal to zero. Thus both input and output will require bias in order to represent negative signals. This bias may be inherent in the characteristics of the gain element in some cases.

### 4. Degradation

In genetic circuits, the active elements typically make the molecules of interest. With only such elements available, concentrations of these molecules may be static or grow but can never decrease. Therefore, to allow signal swings about some mean level, it is essential that the cell provide means to degrade these molecules (for example proteases). This disclosure assumes such processes are indeed present in the cell.

### 5. Input, Feedback and Output

The next consideration is the combination of input and feedback signals at the input of the gain element.

The simplest design is to make input and feedback molecules identical. The gain element could be selected to regulate the total concentration of that molecular species. Thus if the concentration must be constant then when input molecules are added the production of feedback molecules must decrease. Then the degradation processes can degrade the molecules until the concentration is brought back to the normal level. If there is a steady flux of input molecules, then any increase in this flux must lead to a decrease in the flux of feedback molecules by the same amount. Any decrease in this input flux would lead to a matching increase in the flux of feedback molecules. Thus the circuit gain would be −1.

In a more complicated design, the input and feedback molecules are modified to be different yet still capable of binding to the same operator. The goal is to change the relative levels of input and output required to maintain equilibrium. It is analogous to changing the relative values of the resistors in FIG. 1 and thus changing the circuit gain. This may be through modifying the molecules so that they have different affinities to the operator. Alternatively, it could be through changing the degradation rates, for example by adding a tag such as for the ubiquitin degradation pathway for one of them.

Rather than the circuit output being the same as the feedback molecule, circuit output will typically be through another gene attached to the same promoter. This provides flexibility in interfacing the circuit to other genetic circuits, both natural and synthetic. Output and feedback could be separate molecules or could be a fusion protein.

The input molecule is restricted by the choice of gain element. For more general application, this input molecule could be the product of another promoter selected to respond to some other molecule. This might be to permit control by a membrane diffusible molecule, to allow interfacing to a natural cellular circuit, or, to interface to other synthetic genetic circuits.

### 6. Preferred Implementation

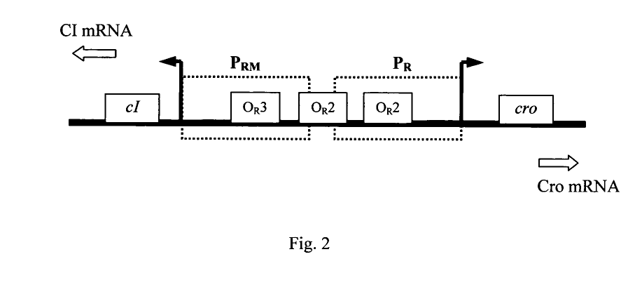

The preferred implementation of the genetic circuit inverting amplifier is designed around the P_{RM} promoter region of bacteriophage λ. As seen in FIG. 2, this promoter is overlapped by the right operator region, which contains three sites: O_{R}1, O_{R}2, and O_{R}3. Repressor protein (CI) dimerizes and binds to each individual operator site. When RNA polymerase binds to P_{RM}, it becomes poised to transcribe the cI gene. If, at that instance, some repressor dimer is already bound to O_{R}1, transcription of cI proceeds at a low level. Repressor dimer bound to O_{R}2 interacts with the polymerase, enhancing its affinity for P_{RM} and increasing transcription of cI by roughly tenfold. In contrast, when repressor dimer is bound to O_{R}3, binding of polymerase to P_{RM} is blocked and transcription is effectively turned off. Thus, the λ repressor protein acts as a regulatory protein whose synthesis is autoregulated through negative feedback. This feature mimics the effect of the feedback loop in an electronic amplifier, making the right operator region of λ DNA a desirable base unit for the design of a genetic amplifier circuit.

Note that with CI bound to O_{R}1 and O_{R}2, binding an additonal CI (to O_{R}3) leads to repression. By design and as confirmed by simulation, the circuit primarily this gain element in the operating region between these two cases wherein the output is a decreasing function of the input.

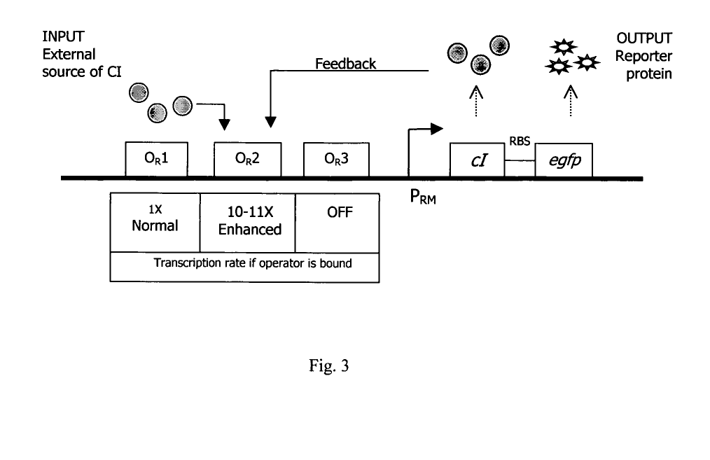

FIG. 3 shows the biological design of the genetic circuit amplifier. The input to the circuit is CI monomer. Feedback of translated CI, which exists in equilibrium with dimer, acts to balance the effect of the input. The feedback and an external source of CI monomer constitute the total input to the gain element.

The output of the system is the expression of GFP protein that is jointly transcribed with CI. Protein expression is an analog output signal that can be quantified experimentally by detecting the fluorescence of the GFP.

The circuit was constructed and tested in the bacterium E. coli. Basic molecular biology techniques as described in cloning manuals [13] were used to construct the amplifier circuit on an E. coli plasmid. All circuit genes and the promoter were isolated by PCR using Taq polymerase. pTrueblue-rop® (Genomics One Corp., QC) was used as the cloning vector. It is a low copy number (15 to 20 copies per cell) plasmid with a transcription terminator at the end of its multiple cloning site that aids in achieving optimized protein expression. The plasmid also carries a selective marker rendering ampicillin resistance. Cloning sites were selected from within the multiple cloning site of the closed plasmid and obtained by digestion with the restriction enzymes NcoI and BamHI.

The circuit was built by cloning amplified segments of DNA, generated by PCR, into plasmids. Cloning is made possible by the addition of unique restriction sites to the 5′ ends of the pair of oligonucleotide primers used in the PCR. The restriction sites are transferred to the ends of every copy of DNA synthesized through each cycle of PCR. Cleavage of these ends with the appropriate restriction enzymes results in strands of DNA with cohesive ends that are tailored to match the sticky ends in an equivalently digested cloning vector.

Primers are designed so that the 3′ end of each primer is the exact complement of 18-22 bases at each flanking end of the target DNA to be copied. The extreme 5′ end of each primer consists of a series of 3 to 10 guanines and cytosines, constituting a “GC clamp”. Many restriction sites fail to cleave recognition sequences located near the ends of the DNA fragments. GC clamps at the 5′ extremes of the primers help to adequately hold together the termini of the DNA fragment and to provide a landing site for the restriction enzymes as it cleaves.

The midsection of the primer is non-annealing to the template DNA and contains the restriction sequence. The midsection may also contain nucleotides that serve as spacers, to ensure that genes cloned side-by-side remain in-frame. In this way, the codons on the mRNA transcripts are read in the correct sense and the proper protein is translated.

The PCR cloning strategy requires that the insert and vector have compatible ends. If only one enzyme is used to cleave the plasmid, the fragment to be inserted should be synthesized from PCR primers that both contain the same restriction site. The fragment then has two possible orientations in which it can attach to the sticky ends of the plasmid. If the PCR primers are designed to contain two different restriction sites and the vector is digested with those two compatible enzymes, then the DNA fragment can be attached into a plasmid with a forced orientation. Whenever possible, the primers should be designed to contain different restriction sites so that the DNA fragment can be directionally attached into a plasmid.

For a plasmid to generate large quantities of a protein, the vector must contain an efficient ribosome binding site (RBS), also known as a Shine-Dalgarno sequence in E. coli. This is a short sequence (5 to 9 nucleotides in length) that interacts with rRNA and serves as an initiation signal for protein synthesis. The sequence AGGAGGA is recognized as a consensus ribosome binding site sequence, but variations on this sequence can produce to stronger ribosome affinities. The RBS sequence is typically located up to 10 bases upstream of the start codon of the protein. The distance between the Shine-Dalgarno sequence and the initiating codon is crucial if maximal expression of the foreign protein is to be achieved [13]. A PCR primer can be engineered for a gene so that it contains a Shine-Dalgarno sequence between the designed restriction sequence and the gene start signal in order to ensure efficient translation of the desired protein.

The P_{RM} promoter overlapped by the right operator region in bacteriophage λ is at the core of the design of the genetic circuit amplifier. The cI gene and the right operator were amplified by PCR from λ DNA template (Amersham Pharmacia Biotech Inc., N.J.). Oligonucleotide primers were synthesized at ACGT Corporation (Toronto). Specifically, the PCR reaction was performed with the forward primer CIORfwd2 and with the reverse primer CIOR_NcoI, yielding the full-length cI gene with the P_{RM} promoter and its operator sites paired with an NcoI restriction recognition sequence at one end and an EcoRI site at the other end. The fragment is 768 nucleotides long in total.

The CIORfwd2 primer is designed to flank the end of the cI gene and has an annealing section that is complimentary to the first 22 bases of the gene. The restriction sequence recognized by the enzyme EcoRI is introduced on the 5′ end of the primer. This region does not anneal to the template λ DNA, but nonetheless gets copied at the end of the gene during each cycle of PCR. A (GC)₄-clamp is positioned at the 5′ extreme end, next to the restriction site to ensure efficient cleavage with the appropriate enzyme. The total length of the primer is 36 nucleotides and its melting temperature is 73.3° C.

The CIOR-NcoI primer anneals to the end of the operator region, overlapping 22 bases. A (GC)₄-clamp is positioned at the 5′ end next to the NcoI restriction site. The total length of the primer is 35 nucleotides and its melting temperature is 74.7° C.

Both EcoRI and NcoI have a cleavage efficiency of 50-100% when just two additional bases are adjacent to the introduced restriction site. Nonetheless, eight additional bases were included in the clamp next to the restriction sites for purposes of maintaining an adequate and sufficiently high melting temperature of the primers—a criterion that affects the quality of the PCR.

Primer design was guided by the OligoAnalyzer courtesy of Integrated DNA Technologies, IO (available for download at http://207.32.43.248/). This software calculates the melting temperature of the primers and determines thermodynamically whether the primer has sequences complimentary within itself. These could lead to the formation of dimers or hairpin loops. As a rule of thumb, the free energy (ΔG) of possible primer configurations should be greater than −2 kcal/mol. Higher values of ΔG indicate a good, stable primer.

An enhanced version of the green fluorescent protein was chosen as the reporter protein for the circuit. Copies of the egfp gene were obtained from the plasmid pEGFP-1 (BD Biosciences Clontech, Calif.) using the polymerase chain reaction. The PCR reaction was performed using the forward primer EGFPfwdRBS and with the reverse primer EGFPr-BamHI. PCR yielded the egfp coding sequence with its Shine-Dalgarno site paired with an EcoRI restriction recognition sequence at one end and a BamHI site at the other end. The fragment is 723 nucleotides long.

The forward primer contains the sequence AGGAGGTAA, a variation on the 7-base consensus RBS sequence, serves as a Shine-Dalgarno sequence upstream of the gene's start codon. A spacer of 7 bases separates the Shine-Dalgarno site from the start codon of egfp. A (GC)₃ clamp is adjacent to the 5′ end of the EcoRI restriction site. The annealing section of the primer is complimentary to 22 bases at the beginning of the gene. The total length of the primer is 47 nucleotides and its melting temperature is 76.3° C.

The EGFPr-BamHI primer is designed to flank the end of the egfp gene and is complimentary to the last 20 bases of the gene. A (GC)₃ clamp is adjacent to the 5′ end of the BamHI restriction site. The total length of the primer is 47 nucleotides and its melting temperature is 76.3° C.

PCR was performed using the non-proofreading Taq DNA polymerase. The reaction was initially denatured at 94° C. for 2 minutes and 30 seconds, followed by 30 cycles of denaturing at 94° C. for 45 seconds, annealing at 58° C. for 45 seconds, and extension at 72° C. for 90 seconds. A final extension period of 10 minutes at 72° C. was inducted before cooling to 4° C. Annealing temperatures were sufficiently close to primer melting temperatures to ensure efficient binding of primers to the template. A summary of these reaction conditions and reagents used in the reaction mixture is included in Appendix B.4 of [18]. The same program was used for both fragments: (1) cI gene with right operator region, and (2) egfp gene.

A MicroSpin™ S-400 HR Column (Amersham Pharmacia Biotech, N.J.) column was used to purify the PCR products, separating them from unused primers, salts, and enzyme remaining in the reaction mixture. This was necessary because impurities lingering from DNA preparations, including unused dNTPs and Taq polymerase from PCR, can partially or completely inhibit restriction enzyme activity later on. The purified PCR products were verified by gel electrophoresis and confirmed by sequencing. As an alternative to the column, gel purification (as will be discussed below) was used in some constructions of the amplifier.

A restriction digest mixture consists of four components: substrate DNA, restriction enzyme, buffer, and water. The amount of DNA depends on the quantity available and the choice of buffer depends on which enzyme is being used. Each enzyme performs optimally in different salt concentrations and pH. Selection of the correct buffer is critical in order to avoid “star activity”, in which the enzymes relax their specificity and cut in sequences that differ from their canonical recognition sites.

Digestion of DNA with two different enzymes may be performed at once only if the buffer used is compatible with both enzymes. The 10× Tango™ buffer (MBI Fermentas Inc, ON) is a universal buffer that has been specifically designed for double digests. For double digests involving any combination of EcoRI, NcoI, and BamHI, reference to the vendor's manual indicates that Tango™ buffer must be used in 2× concentration.

Restriction enzyme activity depends on the nature of the DNA being cleaved. Linear DNA, supercoiled DNA, and the number and type of nucleotides flanking the recognition sequence all challenge the efficiency of cleavage to a variable extent. Recipes for digests are therefore quite flexible. Digests may be accelerated or improved by using more enzyme units. However, some enzymes are sensitive to the amount of glycerol in the digestion mixture. Since restriction enzymes are supplied in 50% glycerol, the enzyme should not exceed 1/10

of the final reaction volume (i.e. no more than 5% glycerol in the digest mixture). Furthermore, large excess of enzyme with respect to DNA may also induce star activity.

Approximately 200 ng of each PCR product (either cI or egfp) was digested in 2× Tango™ buffer. The cI and operator fragment was cut with NcoI and EcoRI enzymes. The egfp fragment was digested with EcoRI and BamHI enzymes. The exact protocol is given in Appendix B.6 of [18].

The digest mixtures were incubated at 37° C. for 2 hours, followed by a short heating period to inactivate the enzymes. The mixture was loaded on a 0.8% agarose gel and viewed on a UV transilluminator. The desired product band was excised with a clean razor blade, taking care to minimize DNA exposure to UV light (<1 minute) in order to prevent photochemical damage. The DNA was recovered from the agarose and purified using the NucleoTrap® Gel Extraction kit (Clontech Laboratories, CA). Gel purification liberates the DNA from unused enzyme, salts, and unwanted cohesive ends that may compete for compatible sticky ends on the cloning vector during ligation later on.

In a similar fashion, 200 ng of the plasmid was digested with enzymes NcoI and BamHI. The mixture was incubated at 37° C. for 6 hours, followed by heat inactivation of the enzymes at 65° C. for 20 minutes. The digested plasmid was treated with Calf Intestinal Alkaline Phosphatase (CIAP). This enzyme catalyzes the removal of 5′-phosphate residues from DNA, improving its efficacy in ligations. The solution containing dephosphoylated and digested plasmid was then loaded entirely on a 0.8% agarose gel. The band at the correct linear length were excised and the DNA was purified using a gel purification kit.

The fragment of cI gene with its right operator and the egfp gene fragment were attached to each other at the EcoRI sticky end by a ligation reaction. Equal volumes of digested and gel purified PCR products were incubated with T4 DNA ligase. The ligation mixture was then used as a template for PCR in order to generate many copies of the in vitro circuit assembly. Primers CIOR-NcoI and EGFPr-BAMHI were used. The PCR cycles were identical to the program used to generate the individual fragments, with the exception that the length of the elongation time increased by one minute to 2.5 minutes (Appendix B.4 of [18]). This is done to accommodate the elongation of a much longer template (˜1500 bp).

Prior to ligation, both the foreign DNA and vector plasmid DNA were digested and gel purified. Excess insert was used in the ligation reaction; an excess foreign DNA to plasmid ratio of 3:1 or 4:1, with at least 20 ng of plasmid, is recommended to achieve the greatest efficiency of ligation. The ligation mixture was incubated at 16° C. for 1 hour and the DNA ligase was heat inactivated prior to transformation. The protocol for ligation may be found in Appendix B.7 of [18]. The ligation product is a plasmid carrying the amplifier circuit and is called pAMP-1 from here forth.

Foreign plasmids can be inserted into bacterial cells that have been rendered competent. Preparation of competent cells is done through treatment with calcium chloride (CaCl₂), as outlined in Appendix B.9 of [18]. It is postulated that the Ca²⁺ ions neutralize the repulsive negative charges of the phosphate backbone of the DNA and the phospholipids in the cell membrane, allowing the DNA to pass through the cell wall and enter the cell. E. coli strain DH5α cells were rendered competent for the uptake and propagation of the plasmid carrying the amplifier circuit.

The plasmid pAMP-1 was suspended in the competent cell/CaCl₂ mixture and incubated on ice for 30 minutes. As little as 1 to 3 μL of plasmid is added to a 50 μL competent cell mixture (Appendix B.10 of [18]). The cell/plasmid suspension is then incubated at 42° C. for exactly 90 seconds and then returned to an ice bath. The rapid change in temperature causes a draft that sweeps the plasmid into the competent cell.

Following thermal shock, the cells undergo a recovery period. The cells are suspended in LB broth and are then incubated at 37° C. for one hour. During this period, the cells stabilize into a normal growth cycle and begin expressing the ampicillin resistance gene carried on the plasmid. The cells are smeared onto an LB-agar plate containing ampicillin at 100 μg/mL. Only those cells that acquired the plasmid during the transformation can survive in this growth medium.

Growing cells that have been transformed with the recombinant plasmid on antibiotic plates should be sufficient to isolate colonies containing the circuit. Unfortunately, false positives sometimes survive on the plates. Screening for false positives is accomplished by blue/white selection of colonies on plates containing X-gal and IPTG.

Recall that the lacZ gene in the lac operon encodes β-galactosidase, an enzyme that metabolizes lactose and analogs of lactose. A colorimetric assay that tests for β-galactosidase activity uses X-gal (5-bromo-4-chloro-3-indolyl-β-d-galactoside) as substrate. β-galactosidase hydrolyzes X-gal to yield an intensely blue by-product. When cells are grown on agar plates containing X-gal and the lacZ gene is being expressed, blue colonies will appear.

The multiple cloning site of cloning vectors such as pTrueBlue-rop® has been engineered so that it is located within the lacZ gene on the plasmid. If no gene is inserted into the MCS, then the lacZ gene gets expressed normally and cells containing this plasmid grow in blue colonies. If a gene is cloned into the MCS, the lacZ gene is inherently broken apart by the insert and can longer be expressed. Therefore, cells containing recombinant plasmids will grow white.

Selected white colonies were picked and transferred to Luria-Bertani (LB) broth containing ampicillin and allowed to grow overnight. Growing cells in liquid media is advantageous because it allows the generation of larger working volumes of the cell containing the circuit. Cells are harvested and the plasmid is isolated using a plasmid mini-preparation protocol (Appendix B.8 of [18]).

The recombinant plasmid was digested with XhoI, an enzyme that has a unique recognition sequence in the part of the multiple cloning site that was undisrupted. This digestion linearizes the plasmid, enabling us to visualize it on an agarose gel. The theoretical linear length of ˜5250 bp (3750 bp from the plasmid and 1500 bp from the circuit) was consistent with the estimated length seen on the gel.

The plasmid was also used as a template for PCR. Primers CIOR-NcoI and EGFPr-BamHI were used to generate copies of the insert. Further confirmation of the cloned circuit was done through sequencing of this PCR product and of the plasmid. The sequence matches 99% of the known sequence of the cI gene. A mixed signal was obtained during the automated sequencing reaction for the egfp gene and so no conclusions can be made about the integrity of the cloned egfp gene.

A second bacterial plasmid, pBT (Stratagene, CA), was selected to produce the input. It releases monomer into the cellular environment, making CI available for the engineered circuit to use. It is a simple plasmid that controls transcription of cI with the P_{lacUV} promoter. This promoter can be chemically induced with IPTG, thereby enabling us to tune the amount of CI input produced. The pBT vector has an origin of replication derived from p15A, making it compatible with the circuit plasmid which has a different ColE1 origin of replication. Together, the plasmids constitute a cis-trans network, since the product of one plasmid (pBT) affects the outcome of gene expression on the second plasmid (pAMP-1).

The input pBT plasmid and the amplifier plasmid pAMP-1 gene were transformed into chemically competent E.coli strain DH5αZ1. This strain was selected because it produces LacI in abundance so as to permit effective use of pBT. Transformed cells were grown overnight at 37° C. in LB broth. Chloramphenicol was added at a concentration of 50 μg/mL and ampicillin was added at a concentration of 100 μg/mL.

The overnight culture was diluted 1:50 into fresh LB medium containing IPTG at the following concentrations: 0-20-200-2000 μM. These cells were allowed to grow for another 3 hours. The cells were then spun down and re-suspended in PBS. Fluorescence was then measured on a BMG Optima microplate fluorometer equipped with filters for GFP. Absorbance at 600 nm was also measured. Fluorescence was the corrected for cell density and background fluorescence. The result, presented in Table 1, indicates an output that decreases with increasing input. Observed non-linearity is likely due to low gain of chosen gain element.

Other variants have been prepared in our laboratory. A version with out the RBS and start codon has been prepared that makes a CI-EGFP hybrid protein has been created. This resulted in a feedback molecule with lower affinity for the operator region and hence higher gain (and non-linearity). A version wherein the feedback CI had a ssrA tag for degradation resulted in a feedback molecule with a shorter half-life than the input molecule; this tag was added through incorporation in a PCR primer in a manner similar to what was discussed above to incorporate restriction sites. Variants have also incorporated the short life aavGFP and asvGFP reporters as provided courtesy of Elowitz[11].

TABLE 1
Output fluorescence as a function of input IPTG concentration.

| IPTG(uM) | EGFP(arbitrary units) |
|---|---|
| 0 | 85405 |
| 20 | 66263 |
| 200 | 58338 |
| 2000 | 44137 |

### 7. Implementation Variations

In addition to the preferred implementation(s) discussed earlier, and simple intuitive variations, the following types of variations on these implementations are explicitly included in this application:

First, variation is possible through substitution of alternative operators, promoters and genes from bacterial, viral and eukaryotic sources. Active proteins, transcription factors, repressors, inducers, enhancers, ligands, reporters and silencers [27] should then be selected to function with these alternatives. Logically and functionally, the substituted units behave the same as those originally specified; however, the active molecules carrying out these functions are different.

Second, variation is possible through the use of any desired or necessary (if required for a specific organism) transformation vector or vectors to carry the circuit or circuits into the organism. These vectors may then effect replication of the circuit(s) if so desired. They may also include, either directly or genetically, additional active machinery such as polymerases and ribosomes to effect or enhance circuit activity.

Third, variation is possible through changing the location of individual operator, promoter and gene structures on the vector or vectors. For example, a circuit composed of two different operons (i.e. promoters and genes) may have both operons placed on the same plasmid or, alternatively, such a circuit may have each of the two operons on a different but compatible plasmid. Further, when certain circuit molecules are capable of logically linking different cells, through for example ligand-receptor interactions or diffusible hormones, then different elements of the circuit may reside in different cells and if necessary, different vectors may be used to effect the respective transformations.

Fourth, variation is possible through the use of components naturally present in the host cell to form part of the circuit. This hybrid circuit is formed of host and vectored components.

### 8. Coupling Internal Circuit Signaling to Desired Cellular or Environmental Signals

The genetic circuits described in this application will typically use molecular species not naturally occurring in the host cell for internal signaling within the genetic circuit itself. This is to minimize potentially harmful unintentional interactions between circuit and cell. However, to effect a desired impact on cellular activities, circuit inputs and/or outputs will need to be coupled to host pathways.

The easiest means to do this is to use elements that naturally couple to host signals. For example, the circuit output may be coupled to the host via having the circuit output promoter coupled to a gene for the host pathway molecule at the desired point of connection. For example, the circuit input may respond to the host via including as the input promoter one whose operator is affected by a repressor, activator, inducer, enhancer, silencer or other transcription factor that is made by the host at the desired point of coupling host output to circuit input. More elaborate is a specially engineered or evolved coupling molecule or coupling molecule complex.

External input to the circuit may be through diffusible molecules based on metabolic substrates such as IPTG, natural diffusible signals such as hormones, or ligand-receptor interaction. Mechanical or electromagnetic (e.g. light [31]) inputs may also be coupled to circuit input via molecular transduction. External output from the circuit may be via a diffusible molecule or a diffusible complex of molecules as in a viral capsid. Output may be through reporters based on fluorescence, chromatographic (e.g. color) change or binding to detectable tag molecules. Transport of molecules in or out of the cell and/or in and out of the nucleus may be through passive diffusion or facilitated by transporter molecules. Access to circuit state as recorded on a nucleic acid, peptide or other non-diffusible molecule may be through destructive lysis of the cell and extraction and identification of signal molecule(s).

### 9. Genetic Circuit Hosts

### a) Prokaryotes

The prior art synthetic genetic circuits were constructed using plasmids as transformation vectors. Promoters and attendant operators used as circuit components were drawn from bacteriophages (bacterial viruses). These vectors and components may be used to construct the circuits presented herein.

In addition, a range of other standard transformation vectors may be used including bacteriophages, cosmids and phagemids to carry the circuit into the host bacterium. Promoters and operators used in circuit construction may also be drawn from other bacterial species. In anticipation of future developments, entirely new synthetic promoters may be created as per research underway by Professor M. Surette at the University of Calgary. Alternatively, genetic regulatory elements from higher organisms may be used provided that the cells may be augmented with additional RNA polymerases and/or ribosomes that can recognize the corresponding transcriptional and translational control sequences. Tuning of circuit interactions (such as an adjustment of reaction rate constants as in repressor operator binding for example) may be accomplished through mutation of corresponding DNA sequences and selection of mutations with desired properties [29-30].

### b) Eukaryotes

Standard transfection vectors may be used to insert the circuit(s) into eukaryotic cells. Standard laboratory transfection vectors may be used. Vectors used in gene therapy [25] such as those based on any of basic retroviruses, lentiviruses, adenoviruses or herpes simplex viruses may be used to introduce the genetic circuit into mammalian cells. Transformation vectors in use for plants may be used to add synthetic genetic circuits to plants. Yeast transformation vectors will allow addition of these circuits to yeast.

For eukaryotic cells, the promoters and transcription factors (including enhancers and silencers as analogs to the activators and repressors of bacterial promoters) that serve as genetic circuit components may come from eukaryotic cells or viruses that infect eukaryotic cells. Typically, the components will have viral sources [26]. For example, Yao [33] has created a controllable promoter structure for eukaryotes by taking the cytomegalovirus (CMV) promoter and adding bacterial tetracycline operator sequences just downstream from it. The result is an expression system that may express any desired gene in eukaryotes with expression level modulated by the tetracycline repressor molecule. Bujard's lab [34] have created controllable expression systems for eukaryotes by creating fusion proteins consisting of a protein that will bind to a DNA sequence of interest and the protein VP16 transcriptional activation domain of Herpes Simplex Virus1. Introduction of this protein leads to the expression of the gene placed after the sequence of interest. Additionally, it is anticipated that new synthetic promoters and transcription factors capable of functioning in eukaryotic cells will be available in the near future. Tuning of circuit interactions, an adjustment of reaction rate constants as in transcription factor binding for example, may be accomplished through mutation of corresponding DNA sequences and selection of mutations with desired properties [29-30]. Less likely sources of components are bacterial or bacteriophage promoters; these will require the introduction into the cells of machinery (polymerases and ribosomes) capable of interpreting the corresponding transcription and translation signal sequences.

Finally, for eukaryotic systems, the circuits may exploit systems for active transport into (and/or out of) the nucleus as part of the circuit itself. Another variation would use mitosis for access to chromatin; this could be for transformation or for synchronizing circuit state with mitosis.

1. A genetic circuit inverting amplifier comprising:
(a) a gain element, comprising nucleic acid constructs with each construct comprising a promoter operationally associated with at least one gene, that produces one or a plurality of molecular species;
(b) an input agent wherein, over an operationally significant range, increasing the level of said input agent decreases production of said one or a plurality of molecular species by said gain element; and
(c) a feedback molecular species wherein
i. said feedback molecular species is one of said one or a plurality of molecular species produced by said gain element; and
ii. over an operationally significant range, increasing the concentration of said feedback molecular species decreases production of said one or a plurality of molecular species by said gain element,
wherein the relation describing the production of said one or a plurality of molecular species as a function of said input agent is substantially determined by the relative activities of said input agent and said feedback molecular species.

2. The inverting amplifier of claim 1, wherein the input agent is electromagnetic energy.

3. The inverting amplifier of claim 1, wherein the input agent is a molecular species.

4. The inverting amplifier of claim 1, wherein the feedback molecular species is a protein.

5. The inverting amplifier of claim 1, wherein said input agent and said feedback molecular species are the same molecular species.

6. The inverting amplifier of claim 5, wherein the gain element is a promoter of bacteriophage λ.

7. A host cell harboring the inverting amplifier of claim 2, 3, 4, or 5.

8. The host cell of claim 7, wherein the host cell is a prokaryotic cell.

9. The host cell of claim 8, wherein the prokaryotic cell is Escherichia coli.

10. The host cell of claim 7, wherein the host cell is an eukaryotic cell.

11. A method of controlling transcription from a promoter in a host cell, the method comprising the steps of:
(i) providing a host cell harboring a recombinant genetic inverting amplifier comprising:
a) a genetic gain element comprising nucleic acid constructs, each construct comprising a promoter operationally associated with at least one gene encoding a control protein, wherein said control proteins interact with said promoters to set the level of production of said proteins, and at least one gene provides a desired nucleic acid transcript or protein, wherein:
1. the production of said proteins is a function of an input agent; and
2. the production of said desired nucleic acid or protein is a decreasing function of the level of said input agent over an operationally significant range of said level of said input agent; and
b) a feedback molecular species whose production is controlled by at least one of the control proteins of said genetic gain element wherein, over an operationally significant range:
1. the production of said feedback molecular species increases when the production of said desired nucleic acid transcript or protein increases;
2. the production of said feedback molecular species decreases when the production of said desired nucleic acid transcript or protein decreases; and
3. the production of said desired nucleic acid or protein is a decreasing function of the concentration of the feedback molecular species over an operationally significant range of that feedback molecular species, and
(ii) providing the input agent,
wherein the relation describing the production of said desired nucleic acid transcript or protein as a function of said input agent is substantially determined by the relative activities of said input agent and said feedback molecular species.

12. The method of claim 11, wherein the input agent is electromagnetic energy.

13. The method of claim 11, wherein the input agent is a molecular species.

14. The method of claim 11, wherein the feedback molecular species is a protein.

15. The method of claim 11, wherein said input agent and said feedback molecular species are the same molecular species.

16. The method of claim 11, wherein the gain element is a promoter of bacteriophage λ.

17. The method of claim 11, wherein the host cell is a prokaryotic cell.

18. The method of claim 11, wherein the prokaryotic cell is Escherichia coli.

19. The method of claim 11, wherein the host cell is an eukaryotic cell.
